(19) United States
(12) Patent Application Publication
(10) Pub. No.: US 2012/0041333 A1
(43) Pub. Date: Feb. 16, 2012

(54) APPARATUS FOR STIMULATING A REFLEXOLOGY POINT ON A FOOT OF A SUBJECT

(76) Inventors: Marie Therese Shelley, Galway (IE); Catherine Ann Quinn, Galway (IE)

(21) Appl. No.: 13/201,436

(22) PCT Filed: Feb. 4, 2010
(86) PCT No.: PCT/EP10/51369

§ 371 Date: Sep. 29, 2011

(30) Foreign Application Priority Data
Feb. 13, 2009 (IE) S2009/0121

(51) Int. Cl. A61B 5/00

(52) U.S. Cl. 600/552

(57) ABSTRACT

An apparatus for stimulating reproductive reflexology points on a foot is described. The apparatus comprises a housing that abuts the foot adjacent one or more sites containing a reflexology point and a vibration device that vibrates portions of the housing to transfer vibrations to stimulate the reproductive reflexology points. The housing abuts the foot on both sides of the ankle at sites containing one or more of a reproductive reflexology point corresponding to one of a uterus, a prostate, ovaries and testes. The housing also abuts the foot of the subject adjacent a reflexology point corresponding to pelvic musculature and vibrates against the heel of the subject to simultaneously co-stimulate the whole heel including the sural and calcaneal branches of posterior tibial nerves.

### CROSS-REFERENCE TO RELATED APPLICATIONS

The present Application is a national stage application from PCT patent application No. PCT/EP2010/051369, filed Feb. 4, 2010, and which claimed priority from Irish patent application No, IE S2009/0121, filed on Feb. 13, 2009, both applications being hereby incorporated by reference herein for all purposes.

### FIELD OF THE INVENTION

The invention relates to a method and apparatus for stimulating a reflexology point on a foot of a subject, and in particular, to a method and apparatus for stimulating the libido and/or wellness of the subject.

### DESCRIPTION OF RELATED ART

Sexual dysfunction in men is generally treated with prescription drugs such as sildenafil citrate (Viagra®), tadalafil (Cialis®) and vardenafii (Levitra®). These pharmaceuticals are particularly used for the treatment of erectile dysfunction. The drugs operate biochemically by increasing blood flow to the penis during arousal. The treatment is effective in many men and accordingly are increasing in popularity. However, as with any drug treatment there may be drug interactions or side effect associated with the therapy. Although a limited amount of research has been carried out, to date, no data is available to suggest that these treatments are suitable for use in female patients presenting with conditions connected to sexual dysfunction. Accordingly, none of these treatments are suitable or approved for use by women who may suffer from sexual dysfunction.

Scientists are clear that men and women have fundamentally different physiological connections between arousal and desire. For men arousal always leads to desire. By improving a man's ability to have erections, drugs such as Viagra® measurably affects male sexual function. Arousal and desire are often disconnected in women. Women, unlike men, need to feel desirable and loved to be ‘turned on’ and in the mood for sex. As a result of these differences, treatment of sexual dysfunction in women and is generally considered more complicated than treatment in men.

In women, it is generally recognised that the female libido is influenced by the emotional well-being and overall self esteem of the women. Clearly then lack of desire or lowering of the libido can result when a woman suffers from emotional issues or other physiological problems associated with emotional well being, such as depression or lack of self esteem, feeling of unattractiveness, relationship problems, abuse, anxiety, stress etc. Physiological factors can also influence the female libido. Mood disorders are closely correlated with low desire and arousal. Women with an anxiety disorder are also more likely to have sexual dysfunction involving desire, arousal, and orgasm. Various fears including fear of letting go, of being vulnerable, of being rejected, or of losing control can all contribute to lessening of the female libido. Sexual desire and arousal may be adversely affected by changes in hormone levels which may occur as a result of use of oral contraceptives, chemotherapy drugs, or existing conditions diseases such as diabetes or high blood pressure. Menopausal or post-menopausal women may have lowered libidos due to hormonal changes associated with menopause. Likewise, sexual desire may be less during pregnancy, or right after childbirth or when a woman is breastfeeding.

Currently antidepressants are one of the prescribed treatments for women suffering from sexual dysfunction. Studies have shown that up to 80% of women with major depression and sexual dysfunction, sexual dysfunction becomes less severe when antidepressants effectively treat the depression. However, sexual dysfunction persists worsens when antidepressants are ineffective. Many women do not wish to take prescription drugs for their condition. Issues with side effects, weight gain and drug interactions may be common. Many women seek alternative or more natural treatments for problems with sexual desire and low libido. Many sufferers turn towards home, herbal or “quack” remedies, often taking the form of unregulated medicinal preparations in an attempt to address their suffering libidos. However, in the majority of cases, the quality, safety and efficacy of such treatments are unknown, consumption of which can involve some risk to the person's health. For a safer, more natural treatment many patients turn to alternative therapies including acupuncture, acupressure and reflexology.

Reflexology is a complementary (alternative medicine) where massaging pressure is applied to parts of the feet (or hands) to promote a beneficial effect on other parts of the body. The technique is considered to induce natural healing. Reflexology is based on the idea that there are zones or reflexes in the feet and hands, which correspond to every part, gland and organ of the body. Application of pressure on these reflexes relieves tension, improves circulation and helps promote the natural function of the related areas of the body. Reflexology centers around the detection of energy pathways in the body through the reflex zones. The energy pathways are assessed for blockage or other disturbances. Disturbances of energy can change the consistency of the feet to feel thickened, spongy, or hard. Applying pressure, (usually with a specific thumb movement exerting a certain amount of pressure) to specific areas in the feet, may elicit pain if there is an energy disturbance in the corresponding body part. Reflexologists believe massaging the area will improve the circulation of energy. Reflexology works best for treating chronic conditions such as pain, menstrual problems, and stress related conditions (Bolting, 1997).

Many existing devices for use in stimulating sexual arousal in women are viewed as sleazy or offensive, such devices include intimate accessories such as vibrators and the like.

Devices involving stimulation of reflexology and/or acupressure points are well known in the art and include the following:

United States Publication No. 2004/054337 discloses a method for performing reflexology to treat conditions. The method comprises steps (a) selecting one or more reflex zones on a patient according to a guide, and (b) topically applying an effective amount of a skin stimulating preparation to the selected reflex zone or reflex zones for an effective length of time with repeat applications as needed. The skin stimulating preparation comprises at least one skin irritant. Preferably, the skin stimulating preparation is provided in the dosage form of plasters. By selecting different reflex zones, many common conditions can be treated by this method.

Great Britain Publication No. 2,286,533 discloses shoes or trainers which contain precisely located magnets. The magnetic field produced by each magnet acts on the reflex zone located in the sole of the human foot. This field is sufficient to stimulate the reflex zone and thus assist to maintain general health and fitness. To maintain stability and for ease of use, the magnets are actually built-in to the sole of the shoe or trainer as opposed to being located in an insole or slip-in shoe support.

French Publication No. 2,823,098 discloses a podology and reflexology element consists of a semi-circular plastic projection, positioned to stimulate the ‘sciatic point’ of the heel. This device is an orthesis for correcting posture and alleviating sciatic pain. The projection has its concave side towards the heel, is integral with the orthesis, and is covered with a surface contact layer.

Chinese Publication No. 2,092,232 relates to a pair of massage shoe pads with air protrusions, which is composed of protruded air sacs that are mutually independent and are not communicated. The upper parts of the protruded air sacs protrude the surfaces of the shoe pads. The protruded air sacs can fully contact soles of feet when the shoe pads are used, and the opening of the soles can produce the function of strong or weak pulse stimulation for promoting the balance of internal secretion, treating diseases and having health care. The utility model which can keep the soles of the feet dry has good effect on chronic disease, such as rheumatic arthritis, sexual hypofunction, etc.

U.S. Pat. No. 6,314,584 discloses a device for ensuring self-massaging of the foot when walking. The device includes a foot-engaging surface comprising a base having raised prints incorporated thereon. The foot-engaging surface is one of a standard type, a medical type, and a paramedical type. The foot-engaging surface is one of a stocking, a tight, a sock and a footpad.

U.S. Pat. No. 4,852,553 discloses a foot zone reflex self-administering therapy apparatus, comprising a display board adapted to display foot reflexology zones corresponding to anatomical areas of the body and stimulating members adapted to be placed on the display board at a zone corresponding to an anatomical area of the body requiring therapy. The user's foot is placed on the display board over the stimulating members in order to apply a kneading, massaging action to the corresponding zone of the user's foot.

United States Patent Application Publication No. 2004/000076 describes a stress reduction kit includes a display package holding a pair of perforated reflex pin holders each with a printed foot, reflexology chart integrally formed thereon. To facilitate creating pressure points on the feet of a user, the stress reduction kit also includes a bag of reflex pins, where each pin is dimensioned to be received in a friction tight fit into any one of the perforations disposed in the reflex pin holders and a set of instructions that guide a user on the placement of individual ones of the reflex pins in respective ones of the reflex pin holders. In accordance with the method of the present invention, when the user inserts the reflex pins in selected ones of the perforations and places the reflex pin holders within his or her shoes, desired body responses are achieved relative to the pressure points created by the pins against the feet of the user.

U.S. Pat. No. 7,264,599 teaches a waterproof bathing slipper having a plurality of nodules for massaging and stimulating specific parts of the soles and sides of the users feet. The present invention includes static and electrically operated pulsating nodules disposed in such a manner as to stimulate specific regions of the foot as applied by the principles of reflexology.

United States Patent Application Publication No. 2009/188129 describes a plantar designed for proprioceptive, exteroceptive, pressoceptive and/or reflexogen stimulation for correcting the muscle-skeletal, myo-facial and/or vascular troubles of the foot sole, comprising a plurality of stimulation members arranged at selected areas of the foot sole in order to perform the stimulation by elastically deforming during the person's deambulation, wherein the stimulation members comprise a plurality of elastically deformable swellings and a plurality of helicoidal compression springs extending in the direction substantially orthogonal to the foot sole.

United States Patent Application Publication No. 2008/161734 discloses a shoe, which incorporates reflexology and acupressure as applied to a foot when wearing the shoe. The shoe has a sole with a battery powered vibration unit that mechanically vibrates a spiked gel insole located near the underside of a foot. The rounded spikes of the insole stimulate the acupressure points of the foot for its relaxation and pleasant feelings in a person. The sole of the shoe has a port for battery charging, a switch to control the vibration, and a charge indicator light at the sole's edge. The sole has a latch to remove the battery from a compartment in the heel area. The vibrating shoe operates while bearing the weight of a person or not. A transmitter remotely activates the vibration unit as a person is standing.

While these devices are suitable for improving the overall health of a subject, a device which is particularly capable of increasing a woman's self esteem while simultaneously promoting positive emotions in the user is not currently available. Such a device would be valuable to promote wellbeing in women with the added advantage of simultaneously increasing sexual desires and increasing female libido without the need for prescription medicines. A device which is suitable for use without supervision of a Reflexologist would be highly desirable. Such a device could be used as needed at home to promote the feeling of wellness and femininity. It would also be desirable to provide a device which can be used to increase feelings of wellbeing and arousal in men in need thereof.

### SUMMARY OF THE INVENTION

According to the present invention, as set out in the appended claims, there is provided an apparatus for stimulating a reflexology point on a foot of a subject, the apparatus comprising:

a housing adapted to abut the foot of the subject at two spaced apart opposite sites thereof, at least one of the sites containing the reflexology point;

a vibrating means for vibrating at least the portions of the housing abutting the site of the foot containing the reflexology point for transferring vibrations to the site thereof to stimulate the reflexology point.

In a preferred embodiment, the reflexology point is a reproductive reflexology point.

In a preferred embodiment, the housing is further adapted to abut the heel of the subject at least one of the sites containing a nerve.

The sciatic nerve is the largest nerve in the body, it is about 2 cm wide at its origin. It passes through the greater sciatic foramen into the buttock then descends through the posterior aspect of the thigh supplying the hamstring muscles. At the level of the middle of the femur it divides to form the tibial branch of the sciatic nerve and the common peroneal nerves.

The tibial nerve descends through the popliteal fossa to the posterior aspect of the leg and passes under the medial malleolus to supply the muscles and skin of the sole of the foot. In the popliteal fossa, the tibial nerve gives off branches to gastrocnemius, popliteus, soleus and plantaris muscles, an articular branch to the knee joint, and a cutaneous branch that will become the sural nerve. The sural nerve is joined by fibres from the common fibular nerve and runs down the calf to supply the lateral side of the foot. The sural nerve is one of the main branches of the tibial nerve. The sural nerve supplies the tissues in the area of the heel, the lateral aspect of the ankle and a part of the dorsum of the foot through branching into the medial calcaneal nerve branches about the heel.

Desirably, the at least one nerve corresponds to the medial calcaneal branches of the posterior tibial nerve in and around the heel. Preferably the nerve is at least one of the medial calcaneal branches of the posterior tibial nerve and sural nerves. Most preferably, both medial calcaneal branches of the posterior tibial nerve and sural nerves are co-stimulated.

It will be understood that use of the term “nerve” herein corresponds to a particular position on the skin of the user substantially overlying a particular nerve in question.

In one embodiment, the housing is adapted to abut the medial calcaneal branches of the posterior tibial nerve in the heel of a foot of a subject. The medial calcaneal branches of the posterior tibial nerve is a branch of the tibial nerve, which is itself, is a branch of the sciatic nerve which supplies the pelvic region.

In one embodiment, the housing is adapted to abut the sural nerve of a foot of a subject.

Most preferably the housing is adapted to abut at least one of the reproductive reflexology points, the medial calcaneal branches of the posterior tibial nerve and the sural nerve of the heel of a subject.

More preferably still the housing is adapted to abut at least one of the reproductive reflexology points and at least one of the medial calcaneal branches of the posterior tibial nerve and the sural nerve of the heel of a subject at more than one point.

Suitably, the housing is further adapted to abut the heel of the subject at least one of the sites containing a nerve. Advantageously, the nerve is at least one of the medial calcaneal branches of the posterior tibial nerve and sural nerves.

More suitably, the device comprises vibrating means for vibrating the portions of the housing abutting the heel of the subject containing at least one nerve for transferring vibrations to the site thereof to stimulate the at least one nerve. Desirably, the at least one nerve corresponds to the calcaneal branches of the posterior tibial nerve in and around the heel and/or the sural nerve.

By housing it is meant a portion of the device that provides a body for accommodating the vibrating device, and includes portions provided for support or gripping the device onto the foot of the user in the correct area for stimulation of the reproductive and/or nerves of the foot about the heel.

By heel it is meant the area of the foot comprising the prominence at the posterior end of the foot. It is based on the projection of the calcaneus bone or heel bone. The heel therefore includes a portion of the sole of the foot and a portion of the lower parts of the back of the ankle supported by the calcaneus bone.

Vibrations can be transferred from the device to the ankle and/or heel of the subject through the housing at the position where it abuts the foot and/or heel.

In a preferred embodiment of the invention the housing is adapted to abut the reproductive reflexology point of a foot of the subject corresponding to the uterus or prostate. It will be appreciated that the reproductive reflexology point corresponds to the uterus in women and that the reproductive reflexology point corresponds to the prostate in men.

In a further embodiment of the invention the housing is adapted to abut the reproductive reflexology point of a foot of the subject corresponding to the ovaries in females or testes in men thereof, it will be further appreciated that the reproductive reflexology point corresponds to the ovaries in women and that the reproductive reflexology point corresponds to the testes in men.

In yet another embodiment of the invention the housing is adapted to abut the foot of the subject adjacent to the reproductive reflexology point of the pelvic musculature.

The inventors of the present device have surprisingly found that concurrent stimulation of reflex points and/nerves on the back and sides of the ankles, together with the heel of the subject promote, in an unexpected synergistical manner, feelings of enhanced well being in the wearer. The user is energized and the libido is effected in a positive manner in a natural way.

The device of the invention has been found to stimulates feelings of wellness in the user through gentle stimulation of the reproductive reflexology points in the foot by providing means for gently vibrating the correct area of the foot of the subject that corresponds to the reflexology point. The reflexology points of reproduction are charted being located on either sides of the ankle. It has been well documented in the literature that stimulation of these points enhances libido.

The synergistic effect is surprising from a reflexologist perspective since there is no teaching or knowledge in the field of reflexology that the calcaneal branches of the tibial nerve (leading to the sciatic nerve) and/or sural nerves (leading to the sciatic) have a role in enhancing libido. The inventiveness of the device lies in its ability to simultaneously result in a synergistic activation of both nerves and reproductive reflexology points operating together within the body to enhance the user's feelings of wellbeing, self esteem and levels of sexual desire and strength of libido.

Few have considered the reflexology point for the pelvic musculature in the heel of the foot. Furthermore there has never been knowledge of a connection between the co-stimulation of the calcaneal branches of the tibial nerve alone or in addition to the stimulation of the sural nerve system in the heel/ankle. The link that gentle stimulation of the calcaneal branches of the tibial nerve alone or with stimulation of the sural nerve together with the reproductive reflex points would lead to enhancement of the libido in a synergistic manner. The apparatus of the invention utilizes reflexology, in combination with stimulation of sciatic nerves in a positive manner to bring result in homeostasis (the state of equilibrium or balance) within the entire body, working holistically for total body wellness. Reflexology is thought to bring balance to the endocrine system, which is in charge of hormone production and regulation, and bring about the relaxation response.

For women and men, over a period of time of using the apparatus, the increased feelings of well being is induced in the user through action on the reproductive reflexology points, calcaneal branches of the tibial nerve, sural nerve, the sciatic nerve and the sacral plexus has a positive, increasing effect on the libido of the subject. Desirably, the libido is enhanced in a natural manner without the need for use of pharmaceuticals or potentially toxic or contaminated herbal or the like. Advantageously, the apparatus of the invention deals with sexual dysfunction in a natural, unobtrusive manner.

In a preferred embodiment the housing of the device is adapted to abut on both sides of the ankle.

The device of the invention can be adapted to stimulate at least one reflexology point on the foot corresponding to the uterus or prostate, ovaries or testes and the reproductive reflexology point of the pelvic musculature.

Preferably, the device is adapted for stimulation of more than one point on the foot.

Preferably, the device is adapted to stimulate, for example, points on the ankle.

Suitably, the device may be adapted to stimulate points on both sides of the ankle.

Desirably, the device may be adapted to stimulate at least one point on the heel. Desirably, the device may be adapted to stimulate the whole heel. Most desirably, the device may be adapted to stimulate the whole heel including the calcaneal branches of the posterior tibial nerve.

It is particularly preferred the device be adapted to stimulate at least one point on the heel corresponding to nerves located in the foot of a subject about the heel region. Advantageously, the device can be adapted to stimulate at least one nerve, wherein the nerve is at least one of the calcaneal branches of the tibial nerve and the sural nerve located in the foot of a subject about the heel region.

Most preferably, the device may be adapted to co-stimulate at least one of the calcaneal branches of the tibial nerve and sural nerves in the foot about the heel.

In a preferred embodiment, the device may be adapted to stimulate all nerves located in the foot. Nerves in the foot include the lateral planter nerve, the medial planter nerve, the sural nerve and the calcaneal branches of the posterior tibial nerve

In a preferred embodiment, the device is adapted to vibrate against the heel of the foot of a subject to simultaneously stimulate both the sural and calcaneal branches of the tibial nerves. Suitably, device is adapted to vibrate against the entire heel of the foot of a subject to simultaneously stimulate the whole heel including the calcaneal branches of the posterior tibial nerve. In other words, stimulation of the points may be achieved by transfer of vibration from the housing to the point(s) and/or nerve(s) in question.

In a particularly preferred embodiment, the device is adapted to co-stimulate at least one of the reproductive reflexology points on the ankle, together with at least one of the areas of the heel of the foot of a subject containing nerves. Suitably, the device stimulates the whole heel, including the calcaneal branches of the posterior tibial nerve. Desirably, the areas of the heel stimulated correspond to the calcaneal branches of the posterior tibial nerve and sural nerve. This is a particularly advantageous arrangement since both the sural and calcaneal branches of the posterior tibial nerve merge further up the leg into the sciatic nerve (the tibial nerve is a branch of the sciatic nerve). The sciatic nerve is the largest nerve in the body, and is attached to the sacral plexus in the abdomen. The sacral plexus stimulates the reproductive organs in the pelvis. The concurrent stimulation of both the sacral plexus system and the reflexology reproductive points in the heel/foot surprisingly produce the desirable effects described herein in the user.

In one embodiment of the invention at least a portion of the housing is of U-shape construction, and preferably, a portion of the housing is adapted to extend across the heel of a foot of the subject, and preferably, the housing extends from the said heel portion on respective opposite sides thereof to the portions which are to abut the respective sites of the foot.

In a preferred embodiment of the invention the housing may be of U-shape construction wherein a portion of the housing is adapted to extend across the heel of a foot of the subject.

In a particularly preferred embodiment, a portion of the housing is adapted to extend around the back of the ankle at a location substantially above the heel of a foot of the subject.

In another embodiment at least one portion of the housing comprises a cup or domed shaped body, which is adapted to accommodate the round of the heel. The round of the heel will be understood by the skilled person to relate to ball of the heel, meaning the fleshy part of the heel that protects the calcaneal bone of the heel. The cup or domed shaped body is adapted to be substantially concave in shape, such that substantially all of the round of the heel may be accommodated therein. Desirably, the cup or domed shaped body is dimensioned such that the skin about the heel of the subject is in intimate inner contact with the cup or domed shaped body on the inner concave side of the body. This facilitates efficient vibration transfer from a vibrating means of the device to the heel of the subject. Preferably, the cup or domed shaped body is disc shaped. Preferably, the cup or domed shaped body of the housing comprises an annular rimmed portion for sealing to the round of the heel of a subject. The annular rimmed portion may additionally comprise a further vibration means for nerve and/or reflex point.

Advantageously, a vibrating means which is provided in an annular shape to contact with the skin about the round of the heel. This is a desirably arrangement since the annular shape allows contact with the entire surface of the round of the heel and therefore can simultaneously stimulate all of the nerves and reflex points about the whole of the heel (including the calcaneal branches of the posterior tibial nerve and sural nerve networks). In particular the annular shaped vibrating means is desirably, since the calcaneal branches of posterior tibial nerve sural nerves at the base of the heel on the sole of the foot and the nerve at the back of the heel may be co-stimulated to give the desired synergistic wellbeing effect in an amplified manner. The annular cup or domed shaped body which may be adapted to accommodate the round of the heel is advantageous for locating the device properly for correct stimulation of the reflex and/or nerve points The cup or domed shaped body also adds to the comfort of wearing the device. Desirably, the cup or domed shaped body comprises a soft cushion for accommodating the round of the heel. Desirably, the cup or domed shaped body comprises a vibrating means.

Ideally, the housing comprises a resilient material to accommodate different widths of feet of different subjects. Desirably, an outer surface of the housing is covered by a cover layer comprising relatively soft material, and preferably, at least the portions of the housing which are to abut the foot of the subject are covered with the cover layer. In one embodiment of the invention the cover layer comprises a fabric material. In another embodiment of the invention, the cover layer comprises a plastics material. Desirably, the housing can be enveloped within a covering. Suitably, the covering may be decorated with various embellishments or attractive aesthetically pleasing designs, etc.

In a preferred embodiment, the housing adapted to abut the foot of the subject at two spaced apart opposites sites thereof may comprise at least one arm member which may grip the foot of the subject at a position just below the ankle bone. Desirably, the at least one arm member contacts the subject at least one of the sites of the reproductive reflex and a nerve as described herein. In a particularly preferred embodiment a pair of arm members are provided. Suitably, the at least one arm member may be made of a flexible material. A flexible material is desirable since it will allow the arm member(s) to be adjusted to sit more loosely, or more snuggly as the user desires. Preferably, the device comprises a pair of flexible arm members for gripping the foot of the subject below the ankle. Such arm members are desirable since they allow for a more aesthetically pleasing design, lighter and less cumbersome design. A subject is more likely to wear such a device as compared to a more bulky housing. In a particularly preferred embodiment, the device of the present invention comprises two vibrating arms adapted for wrapping snugly around both sides of the ankle. The vibrations from the arms stimulate both the reflexology points of reproduction as well as the calcaneal branches of the tibial nerve (lead to gentle stimulation of the sciatic) and/or sural nerve, located in the ankles and heels of both feet (soles and back of heels).

While the housing has been described as being of a resilient material to facilitate drawing of the side portions thereof towards each other to accommodate different sizes of feet, it will be readily apparent to those skilled in the art that this, while it is preferable, is not essential, since the apparatus could be provided in many different width fittings. Indeed, even where the housing is provided to have some resilience, a number of housings of different width fitting may also be provided, for example, in sizes of small, medium and large.

Preferably, at least one arm members may comprise vibrating means for vibrating the arm members at the site of the foot containing the reproductive reflexology point for transferring vibrations to the sites thereof to stimulate the reproductive reflexology point and/or the calcaneal branches of the tibial nerve and sural nerve. In particular vibrating means in the arm member contacting the ankle transfers vibration to the sural nerve.

In one embodiment of the invention a heating means is provided for heating the housing at least at the portions thereof abutting the sites of the foot of the subject. Heating means is desirable since application of heat to the reflexes and/or nerves in question may enhance the stimulatory effect and synergistic results. It may also be more soothing to the user. Having the option of a heated apparatus may encourage the user to use the apparatus more frequently so that the optimum beneficial effects may be achieved.

In another embodiment of the invention the housing is a hollow housing, and preferably, the vibrating means comprises a vibrator located in the housing. Advantageously, the vibrator is an electrically powered vibrator. A hollow housing is desirable, since it will provide a lighter device, which may be more comfortably worn by the user.

In another embodiment of the invention the heating means is located in the housing, and preferably, is provided by an electrically powered heater, and advantageously, a heat transfer means is provided for transferring heat from the heating means to the respective portions of the housing abutting the sites of the foot. Alternatively, the vibrating means and the heating means are adapted to be powered by a mains electricity supply, and preferably, by the mains electricity supply through a step-down transformer.

While the apparatus has been described as being provided with a heater for heating the housing, it is envisaged that the apparatus may be provided without a heater.

While the apparatus has been described as comprising a hollow housing, in certain cases, the housing may be of a solid construction, and the vibrator may be embedded in or mounted exteriorly on the housing, and additionally, the heater may be embedded in or mounted exteriorly on the housing. It is also envisaged that in certain cases the battery may be provided externally of the housing, and a single battery may be provided for operating the vibrator and the heater of each apparatus of a pair of apparatus.

While the apparatus according to the invention has been described as comprising a housing of a specific shape and construction, it will be readily apparent to those skilled in the art that the housing of the apparatus may be of any other suitable size, shape or configuration, which would be suitable for abutting the sites containing the reproductive reflexology points in the foot of a subject.

In another embodiment of the invention a battery is provided for powering the vibrating means and the heating means, and preferably, the battery is housed within the housing. This arrangement lends itself to a more aesthetically pleasing device and the wearer is not restricted to using the device near a power socket. A battery operated device enables the device to be worn while the user is relaxing. A battery operated device is more convenient and enables the subject to wear the device more frequently so as to receive the full benefits obtainable from use of the device. Desirably, the battery is a rechargeable battery and can be recharged separately as required.

It is also envisaged that instead of being powered by a battery, the apparatus may be powered by mains electricity, in which case. It is envisaged that the mains electricity would be supplied to the respective apparatus through a step-down transformer, and typically, the transformer would be adapted to output a supply voltage to the respective apparatus at a safe level, typically, up to 12 volts.

In a preferred embodiment, a timer may be provided. Desirably, the time can be set to produce a pattern of vibration or continuous vibration for a predetermined period of time. Suitably, in an embodiment comprising a heater, the timer may be linked to the heating means to provide heat in a predetermined manner.

In another embodiment of the invention a control circuit is provided for controlling the operation of the heating means, and preferably, the control circuit activates the heating means to initially bring the temperature of the housing adjacent the portions thereof to abut the sites of the foot of the subject to a predefined temperature prior to placing the device on the foot, and preferably, a sensor is provided for monitoring the temperature of the respective portions of the housing which are to abut the sites of the foot of the subject.

In another embodiment of the invention the control circuit is responsive to the temperature sensor for either deactivating the heating element on the temperature of the housing adjacent the portions thereof to abut the sites of the foot when the temperature of the said portions exceeds the predefined temperature.

In another embodiment of the invention the control circuit is responsive to the sensor determining that the temperature of the portions of the housing to abut the sites of the foot reaching the predefined temperature for operating the heating means at the lower level for maintaining the temperature at the portions of the housings to abut the sites of the foot for maintaining the temperature thereof at the predefined temperature.

While the control circuit has been described as comprising a timer and a temperature sensor, while this is advantageous, it is not essential.

It has been found that approximately a five to fifteen minute period during which the reproductive reflexology points and/or nerve(s) are subjected to vibrations is sufficient for significantly raising the libido of the subject. Most preferably, the device may be used for eight to ten minutes to provide the beneficial results. Most preferably still the device may be worn for approximately ten minutes to observe the desired effect.

In another embodiment of the invention a securing means is provided for securing the housing on a foot of the subject, and preferably, the securing means comprises a strap adapted to extend just beneath the ankle and above the bridge of the foot from one end of the housing to the other. Advantageously, the securing means is adjustable, so that the pressure applied to the reflexes/nerves is adjustable by tightening/loosening the straps.

In an embodiment where the housing comprises a pair of arm members which are adapted to sit in position over the reproductive reflex under the ankle bone, the arm members may be made of a flexible material. Preferably the flexible arms are resilient. Advantageously, resilient arm members can be urged towards each other in such a manner as to hold the device in the correct position on the foot of the subject. Such an arrangement is useful since further securing means are not required. Advantageously, the arm members function as securing means. Preferably, the device may be provided with additional securing means, such as securing straps or securing means in the form of a sock type arrangement. Such a sock could be elastic in nature and sized to fit various sizes of feet.

In one embodiment of the invention a pair of the apparatus is provided, one apparatus being provided for each foot of the subject, it has been found that the best results are achieved when a device is worn on both feet of a user at the same time.

It will also be appreciated that instead of providing a pair of apparatus, a single apparatus may be provided, and in which case, the feet of the subject would be sequentially subjected to vibration by the apparatus.

The invention also provides a method for stimulating a reproductive reflexology point of a foot of a subject, the method comprising vibrating a site of the foot of the subject containing the reproductive reflexology point.

Preferably, the site is vibrated by applying a vibrating means to the site. Advantageously, the vibrating means is heated prior to applying the vibrating means to the site of the foot of the subject.

In one embodiment of the invention the reproductive reflexology point is stimulated using the apparatus according to the invention.

Additionally the invention provides a method for improving the libido of a subject by stimulating a reproductive reflexology point of a foot of the subject by vibrating a site of the foot of the subject containing the reproductive reflexology point, and preferably, the reproductive reflexology point is stimulated using the apparatus according to the invention.

### BRIEF DESCRIPTION OF THE DRAWINGS

The invention will be more clearly understood from the following description of ah embodiment thereof, which is given by way of example, only, with reference to the accompanying drawings, in which:

FIG. 1 is a perspective view of apparatus according to the invention for stimulating a reproductive reflexology point on a foot of a subject,

FIG. 2 is a front elevational view of the apparatus of FIG. 1,

FIG. 3 is a side elevational view of the apparatus of FIG. 1,

FIG. 4 is a transverse cross-sectional side elevational view of the apparatus of FIG. 1 on the line V-V of FIG. 2,

FIG. 5 is a block representation of an electrical circuit of the apparatus of FIG. 1,

FIG. 6 is a side elevational view of the apparatus of FIG. 1 in use on a foot of a subject illustrated from the outside of the foot,

FIG. 7 is a side elevational view of the apparatus of FIG. 1 in use on a foot of a subject illustrated from the inside of the foot, and

FIG. 8 is a diagrammatic view of a pair of the apparatus of FIG. 1 in use on respective feet of a subject,

FIG. 9 is a transverse cross sectional view of a preferred embodiment of an apparatus according to the invention on the foot of a subject, along line V-V of FIG. 10,

FIG. 10 is a rear elevational view of the preferred embodiment of the apparatus of FIG. 9 showing the part of the device which stimulates the heel of the wearer,

FIG. 11 is a top elevation view of a preferred embodiment of the apparatus of the invention showing the position of the operating switches,

FIG. 12 is a block representation of the electrical circuit, of the preferred embodiment of the invention shown in FIG. 9,

FIG. 13 is a side elevational view of the outside of foot of preferred embodiment of the apparatus of the invention shown in FIG. 9 worn on the subject's right foot,

FIG. 14 is a side elevational view of the inside of foot of preferred embodiment of the apparatus of the invention shown in FIG. 9 worn on the subject's right foot,

FIG. 15 is a rear perspective view of a pair of apparatus of FIG. 9 in use on respective feet of a subject,

FIG. 16 is a front elevational view of the apparatus of FIG. 9 showing the arm members in the open position,

FIG. 17 is a front elevational view of the apparatus of FIG. 9 showing the arm members in the closed position,

FIG. 18 is a side elevation view of the apparatus of FIG. 9 showing the dip in the arm members to ensure contact with the reflexology points on the ankle,

FIG. 19 is a rendered side elevation view of the apparatus of FIG. 9 without the holding strap.

FIG. 20 is a rendered side elevation view of the apparatus of FIG. 19 without the holding strap on the foot of a subject,

FIG. 21 shows a rendered side elevational view of the embodiment of the apparatus of FIG. 9 having a decorative covering over the device.

### DETAILED DESCRIPTION OF THE INVENTION

The device is directed towards both women and men to assist them in relaxing, recharging, rejuvenating their sense of well being while stimulating the sexual organs and in a natural way, increases the innate sexual desire of the woman and men to result in an increase in feelings of wellbeing and increased libido. The device does not necessarily have to result in sexual relations, as can used to simply make the woman or man feel good within herself/himself.

The device is superior to existing reflexology products since it focuses exclusively on stimulation of reflexes and/or nerves located on the ankles and heels of the wearer. The other reflexology points on the soles and on the top of both feet are not taken into consideration as the device simply needs to act on the ankles and heels to provide a rejuvenating effect to the libido of the subject.

There are 31 pairs of spinal nerves that feed messages to and from the spinal cord, which subsequently relays impulses to the brain. Each group of nerves are named and grouped accordingly to the vertebrae to which they are associated. Thus the sacral plexus are in the sacral area of the pelvis otherwise known as the reproductive area of the pelvis. The sacral plexus is part of a sheaf of nerves which resemble a horse's tail (Cauda equina). This bundle of nerves branch off from the level of the first lumbar vertebrae where the spinal cord ends. The sacral plexus divides into a number of branches supplying the muscles and skin of the pelvic floor as well as supplying the sciatic nerve.

The sciatic nerve supplies nearly the whole of the skin of the leg, the muscles of the back of the thigh, and those of the leg and foot. The sciatic nerve is very important in the context of this invention, since not only is it the largest nerve in the body, measuring 2 cm in breath, it is also the continuation of the flattened band of the sacral plexus. The sacral plexus stimulates the pelvis and all the organs therein including the organs of reproduction.

The sciatic nerve divides into two large branches called the tibial nerve and the common peroneal nerves. This division may take place at any point between the sacral plexus and the lower third of the thigh. The tibial nerve is the larger of the two branches, which runs along the back of the leg and the heel and is the most important in the context of the present invention since it supplies the heel. The branches of the tibial nerve include: articular—knee joint; muscular—muscles; medial sural cutaneous—joins with sural nerve; medial calcaneal—skin of heel; medial and lateral planter—metatarsal bones and the lateral planter.

The sural nerve runs along lateral side of foot to the little toe. The arm members of the device of the invention also hit the sural nerve. However, the most important part of the device is that directed towards the heel and the stimulation of the calcaneal nerve.

Stimulation of the reflexology points of reproduction opens the energy pathways. Stimulation of the medial calcaneal branches of the sural nerve and/or tibial nerves send impulses up through the tibial and sural nerve towards the sciatic nerve, which in turn stimulate the sacral plexus, pelvic musculature and reproductive organs which are suspended in the pelvis.

Concurrent stimulation of the reproductive reflexology points, the tibial nerve and the sural nerve (through the medial calcaneal branches) has a surprising synergic effect, wherein co-stimulation of each enhances the effectiveness of the other. This is the true strength and originality of this invention.

The apparatus of the invention is thought to operate by co-stimulation of reproductive reflexology points of the foot/heel together with the sural nerve (through the medial calcaneal branches) in the area of the heel and lateral part of the ankle, and the tibial nerve on the sole of the foot and under the ankle.

The sacral plexus passes in front of the sacrum and is distributed through regions of the buttocks. Among the important nerves arising from it are the superior gluteal nerve and the inferior gluteal. Abductors of the thigh and extensors of the thigh are innervated by these two nerves respectively.

The sacral plexus gives rise to the largest nerve in the body, the sciatic nerve. This is the thickest and longest nerve in the body. The sciatic nerve extends from the pelvic area in the body to the heel of the foot. The sciatic nerve splits to form the tibial nerve and the perineal nerve. The tibial nerve supplies the gastrocnemius and other muscles of the lower leg, before branching in the foot to the medial plantar nerve and the lateral planter nerve and the calcaneal branches of the tibial nerve. The medial sural cutaneous nerve arises from the tibial nerve and supplies the skin of the medial portion of the lower leg, while the lateral sural curtaneous arises from the perineal nerve to supply the lateral portion of the skin.

The sacral plexus has an important connection to the lumbar plexus and are sometimes considered together as the lumbosacral plexus. The lumbarsacral plexus supplies the anterior lacteral abdominal walls as well as the genital organs and thighs. Branches of this plexus, called the ilohypogasticnerve supplies the genital organs in the male and female as well as aspects of the thighs. The labia of the female are also supplied by this nerve. In males the cremaster muscle of the male genital organs receives impulses from another branch, called the genitofemoral nerve.

The lumbarsacral plexus also gives rise to the pudendal nerve which extends to the perineum and supplies many regions of the male and female external genital organs.

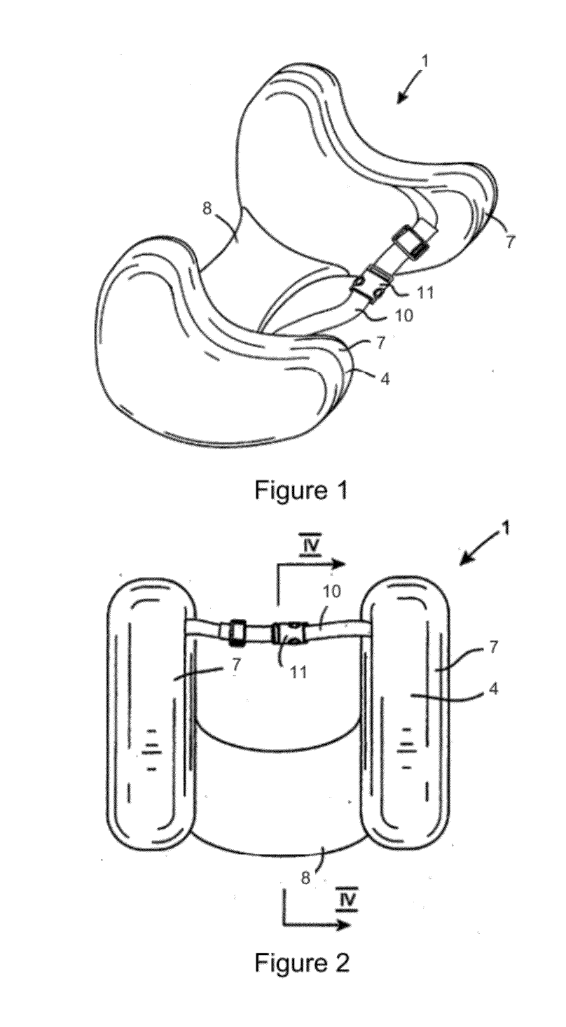

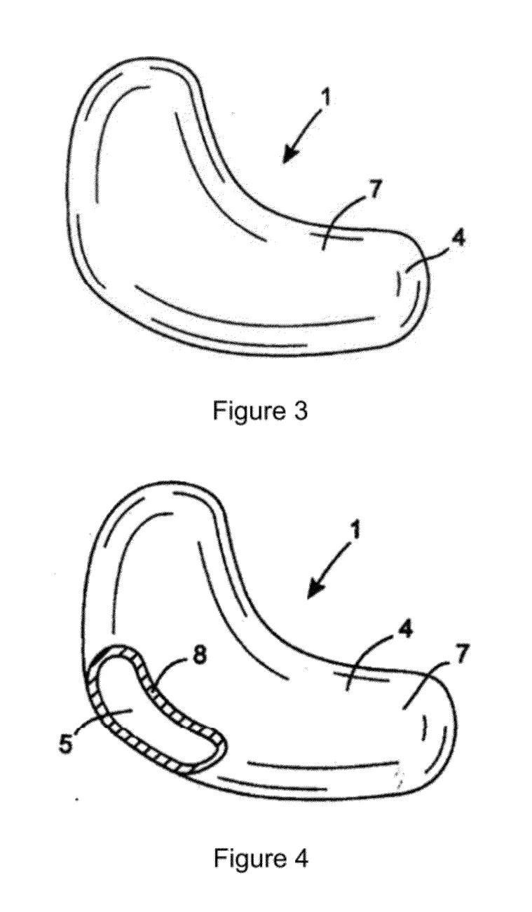

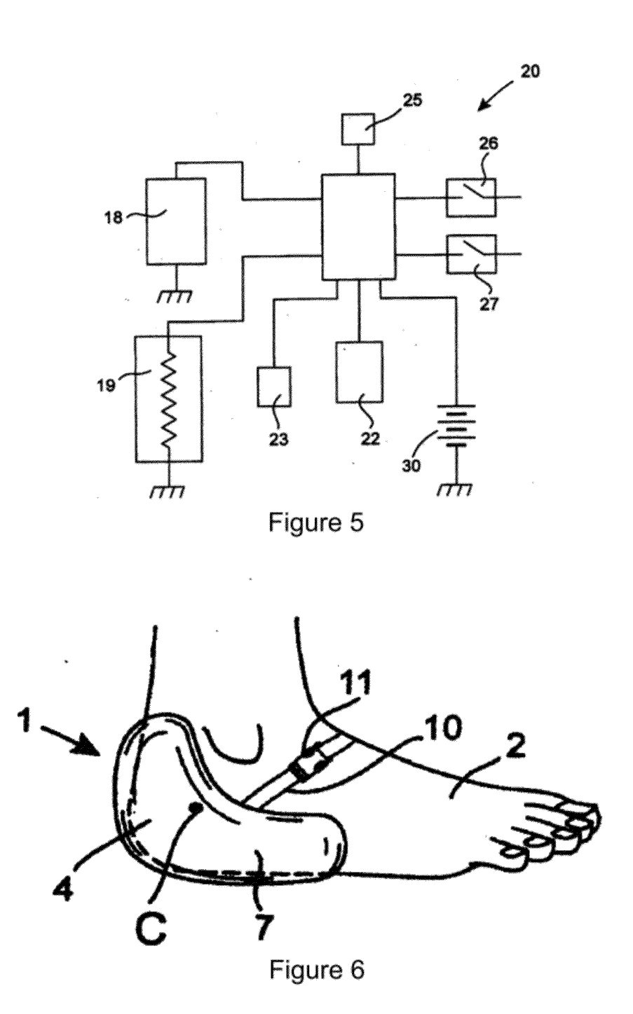

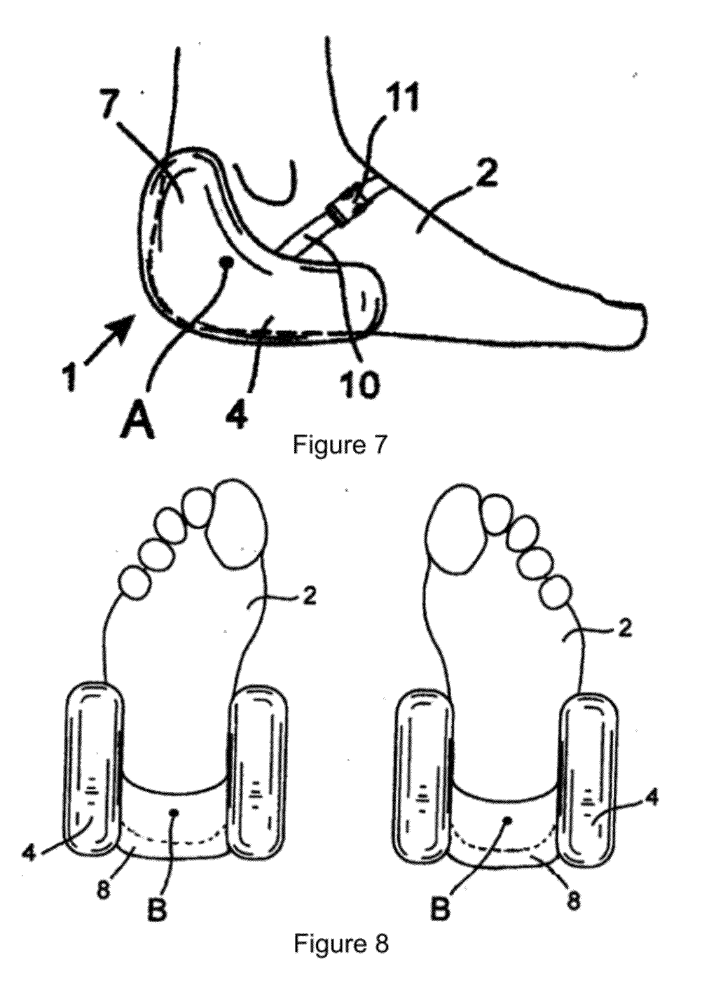

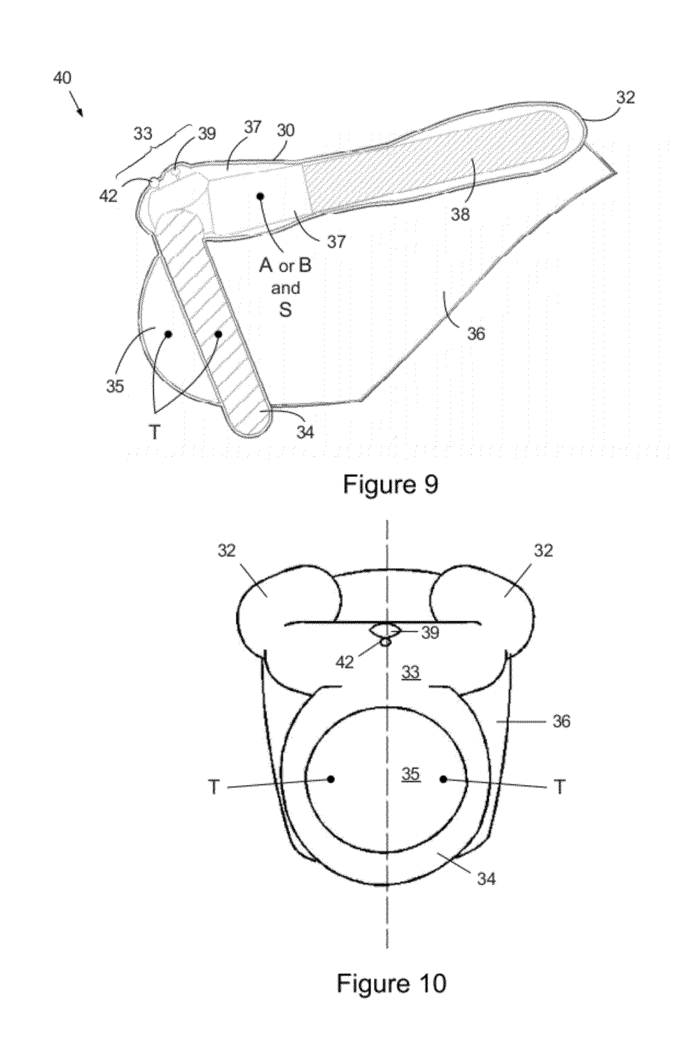

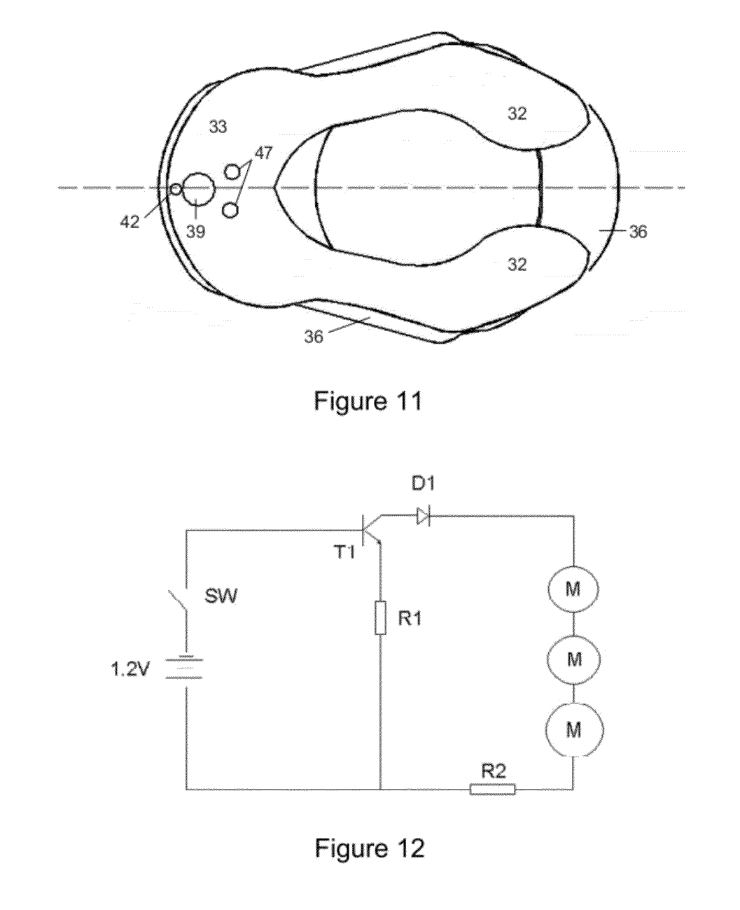

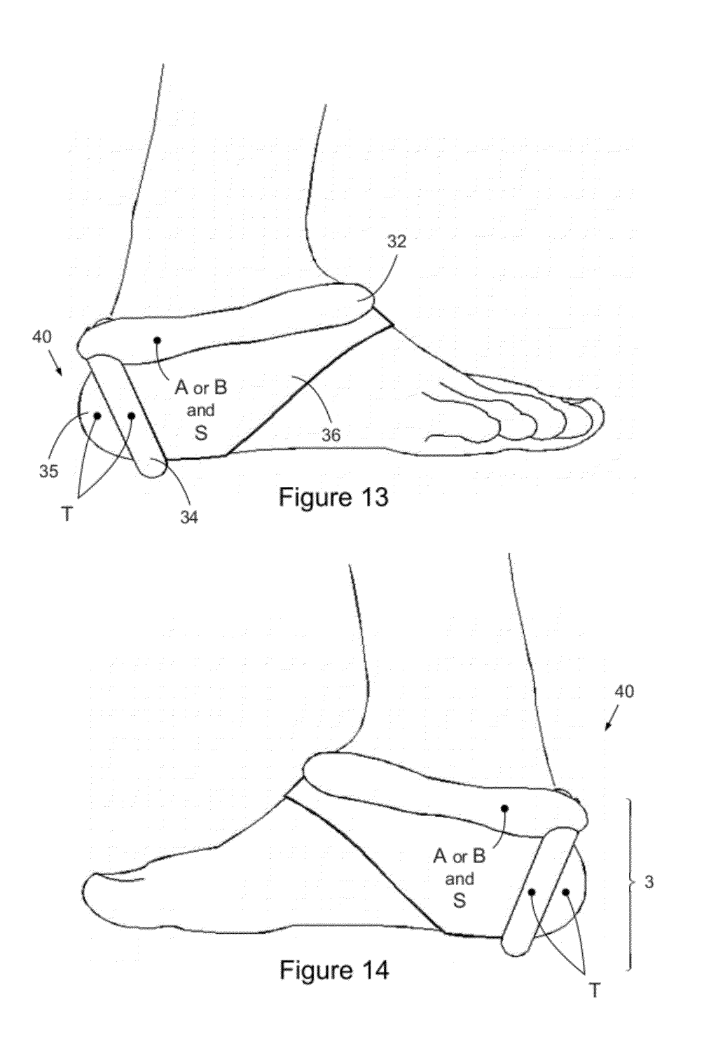

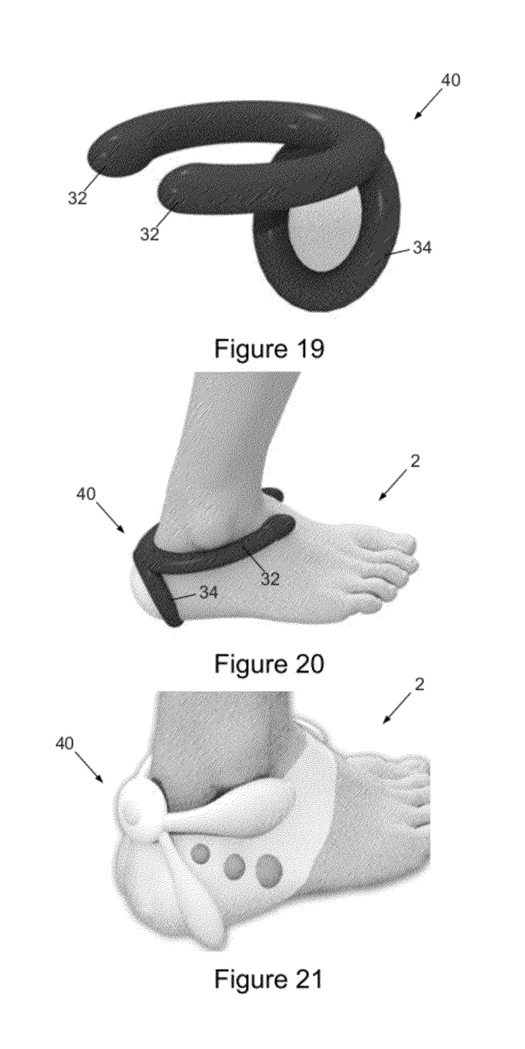

Referring to the drawings and in particular FIGS. 1 to 8, there is illustrated general apparatus according to the invention, indicated generally by the reference numeral 1, for stimulating three reflexology points of reproduction on the foot 2 of a subject in order to stimulate the libido of the subject. FIGS. 9 to 21 illustrate a preferred embodiment of the apparatus of the invention as generally described in FIGS. 1 to 8. The reproductive reflexology points are identified in FIGS. 6 to 8 and FIGS. 13 and 14 of preferred embodiment of the apparatus of the invention. Reflexology point A which corresponds to the uterus or the prostate, the reflexology point B which corresponds to the pelvic musculature, and the reflexology point C which corresponds to the ovary or testes. Nerve point T corresponds to area of the foot where the calcaneal branches of the posterior tibial nerve may be stimulated while nerve point S corresponds to the area of the foot where the branches of sural nerve may be stimulated to induce the desired effect.

The apparatus 1 and preferred embodiment, apparatus 40, may be provided in pairs, one apparatus for each foot 2, and the apparatus for the respective feet 2 are similar to each other.

Each apparatus 1 comprises a housing 4 of hollow construction defining a hollow interior region 5, and typically, the housing 4 will be formed as to half shells secured together. The housing 4 comprises two spaced apart side portions 4 which are joined by a central portion 8. The side portions 7 are adapted for embracing opposite sides of the heel of the foot below the ankle thereof, and the central portion 8 is provided to extend slightly beneath and behind the heel of the foot 2. A securing means, namely, a strap 10 which is provided in two halves joined by a buckle 11 extends from one side portion 7 to the other side portion 7 for facilitating securing the housing 4 to the foot 2.

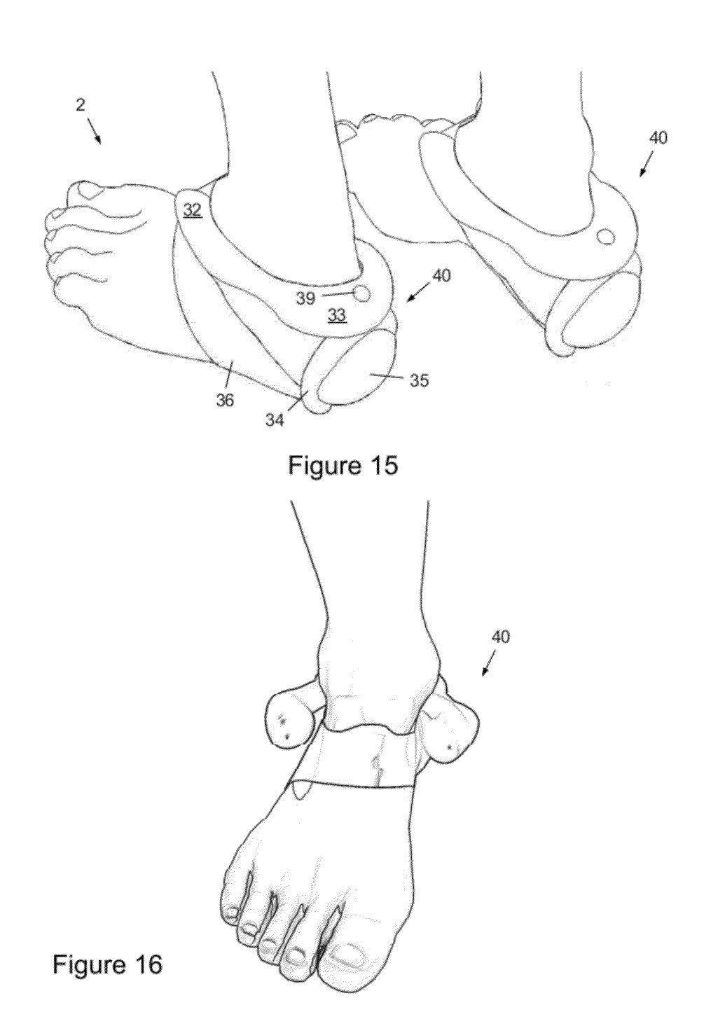

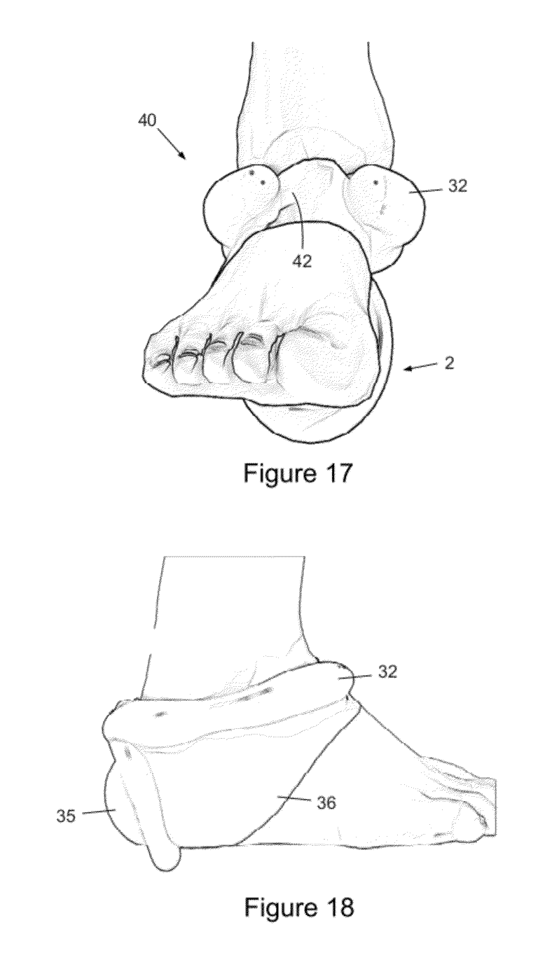

Each apparatus 40 comprises housing 30 of hollow constructions defining hollow interior region 31, the housing being formed from two halves assembled together. Housing 30 of apparatus 40 comprises two spaced apart arm members 32 for gripping opposite sides of the foot 2 below the anklebone protrusions (shown in FIG. 16 and FIG. 17). Arm members 32 of apparatus 40 are joined by a connecting central portion 33 which rests in the inwardly curved portion of the heel 3 of the foot 2 of a subject. Annular shaped ring 34 is made from a solid plastic material to facilitate transfer of vibrations from vibrator 37 (shown in FIG. 9) through the annular ring 34 and onwards to vibratable diaphram 35 to produce vibrations at the heel. Annular ring 34 is suitably dimensioned to substantially accommodate the round of the heel 3. Furthermore, the surface of this portion of the heel is abutted by the vibratable diaphragm 35. Apparatus 40 is provided with a securing sock 36 into which the foot 2 of a user may be inserted to hold apparatus 40 in place in the correct position. Securing sock 36 may be adjusted for comfort of fit by Velcro straps 42 (illustrated in FIG. 15).

The housing 4 and housing 30 may be of any suitable material, and may be of metal, but more typically will be of a plastics material, and the material of the housing 4 will be chosen, and the central portions 8 and 33 will be dimensioned so that the central portions 8 and 33 will be slightly resilient for facilitating drawing of the side portions 7 of apparatus 1 and arm members 32 of apparatus 40, towards each other for accommodating different widths of heels of the feet of different subjects. At least a portion of arm members 32 of apparatus 40 are typically of a flexible material such as plastic or rubber to facilitate bending to fit around the front on the ankle for fit and to position the arm members 32 in the correct positions. The side portions 7 of apparatus 1 and arm members 32 of apparatus 40 are sized to embrace the opposite sides of the foot 2 adjacent the heel 3 and beneath the ankle, and to abut the respective opposite sites of the foot adjacent the heel thereof which contain the respective reproductive reflexology points A and C corresponding to the uterus or prostate and the ovary or testes, respectively. The central potions 8 of apparatus 1, and annular ring 34, of apparatuses 1 and 2, respectively are sized to abut the site beneath the heel 3 which contains the reproductive reflexology point corresponding to the pelvic musculature.

A vibrating means, namely, a vibrator 18 is located in the hollow interior region 5 of the central portion 8 of the housing 4 of apparatus 1 for vibrating the housing 4 in order to transmit vibrations to the reproductive reflexology points A, B and C (FIG. 6 to 14).

Apparatus 40 has vibrator 37 for vibrating the housing 30 of apparatus 40 and which is located in the hollow interior region 31 of central portion 33 of housing 30. Arm members 32 have battery 38 in the hollow interior of at least one of arm members 32. The battery 38 is typically a rechargeable battery. Vibrator 37 of apparatus 40 stimulates the reproductive reflexology points A, B and the sural nerve system.

When apparatus 40 is correctly fitted, vibrator 37 rests over the location S corresponding to the area of the foot overlying of a branching region of the sural nerve S. Vibrations from vibrator 37 travel through the solid material of annular ring 34 of apparatus 40 and onto diaphragm 35 to vibrate on and around the heel at a location T which correspond to the area of the foot overlying the branches of the medial calcaneal nerve. The medial calcaneal nerve is a branch of the tibial nerve.

A heating means provided by an electrically powered resistive heater 19 is also located in the hollow interior region 5 of the housing 4 of apparatus 1 for raising the temperature of the housing 4 to a predefined temperature, which in this embodiment of the invention is approximately 42°, so that the temperature of the housing is approximately 5° C. above body heat.

A control circuit 20 is located in the hollow interior region 5 of housing 4 of apparatus 1 for controlling the operation of the vibrator 18 and the heater 19.

For apparatus 1, a timer 22 located in the hollow interior region 5, times the period of time during which the vibrator 18 should be operable.

For apparatus 40 a timer 41 is located within hollow interior region 31 of housing 30 and determines the period of time for which vibrator 37 operates.

For apparatus 1, a temperature sensor 23 also located in the hollow interior region 5 and monitors the temperature of the housing 4, and the control circuit 20 is responsive to signals from the temperature sensor 23 for controlling the operation of the heater 19. An alerting means, in this embodiment of the invention provided by piezoelectric sounder 25 located in the housing 4 of apparatus 1 outputs an audible signal under the control of the control circuit 20 on the temperature sensor 23 detecting that the temperature of the housing 4 is at the predefined temperature. A pair of button-operated switches 26 and 27 located in the housing 4 are provided for activating the control circuit 20 to operate the vibrator 18 and the heater 19 respectively. A battery 29 located in the hollow interior region 5 of the housing 4 is provided for powering the control circuit 20, the vibrator 18 and the heater 19.

Although not illustrated in FIG. 1 to 8, the housing 4 of each apparatus 1 is covered by a cover layer of relatively soft material, which in this embodiment of the invention is a soft textile material to provide comfort to the subject. FIG. 20 illustrates a typical cover X suitable for use with apparatus 40 of the invention.

In use, a pair of the apparatus 1 and 40 are provided, one for each foot 2 of the subject. Initially when it is desired to stimulate the libido, a subject operates the button switch 17 of apparatus 1 and a switch 39 of apparatus 40 respectively. This in turn which in turn operates the control circuit 20 of apparatus 1 for activating the heater 19. On activation of the switch 27 the control circuit 20 monitors the temperature sensor 23 of the corresponding apparatus, and on the temperature of the housing 4 reaching the predefined temperature of 42° C., the control circuit 20 activates the piezoelectric sounder 25 to output an audible sound in order to indicate to the user that the housing 4 of the corresponding apparatus 1 is at the predefined temperature. Once the temperature of the housing 4 has been raised to the predefined temperature, the control circuit 20 operates the heater of the corresponding apparatus 1 in order to maintain the temperature of the housing at the predefined temperature of 42° C.

Activating switch 39 of apparatus 40 triggers the circuit of FIG. 12 which comprises an on/off timer 41 (FIG. 9) to control the length of time of operation of vibrator 37. Typically operation of the apparatus 40 for a period of about 10 minutes is sufficient to provide the beneficial effect. Apparatus 40 of the invention may have LED 42 for indicating when the device is operating and/or the battery 38 is suitably charged.

On the housing 4 of each apparatus 1 being raised to the predefined temperature, the housings 4 are secured to the heel of the subject by the corresponding straps 10 and buckles 11. The housings 4 are secured to the heel of the subject so that the respective side portions 7 thereof abut the sites of the feet containing the reproductive reflexology points A and C corresponding to the uterus/prostate and the ovary/testes, respectively, and the central portion 8 of each housing 4 abuts the reproductive reflexology point B corresponding to the pelvic musculature.

With the two apparatus 1 and/or 40 attached to the respective feet of the subject, the subject then activates the button switch 26 of each apparatus 1 and/or switch 39 of apparatus 40, which in turn activates the control circuits 20 of apparatus 1 and/or control circuit 60 (see FIG. 12) to operate the vibrator 18 of the respective apparatus 1 and/or vibrator 37 of apparatus 40. Simultaneously with activating the vibrators 18, the respective control circuits 20 operate the corresponding timers 22 for timing a five-minute period during which the vibrators 18 should operate. On the respective timers 22 having timed the five-minute period, the control circuits 20 of the respective apparatus 1 deactivate the corresponding vibrator 18. Vibrator 37 of apparatus 40 is designed to operate for ten-minute periods.

The invention is not limited to the embodiment herein before described, which may be varied in construction and detail.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

1. An apparatus for stimulating a reflexology point on a foot of a subject, the apparatus comprising:
a housing adapted to abut the foot of the subject at two spaced apart opposite sites thereof, at least one of the sites containing the reflexology point;
a vibrating means for vibrating at least the portions of the housing abutting the site of the foot containing the reflexology point for transferring vibrations to the site to stimulate the reflexology point.

2. An apparatus according to claim 1 wherein the reflexology point is a reproductive reflexology point.

3. An apparatus according to claim 1 in which the housing is adapted to abut the reproductive reflexology point of a foot of the subject corresponding to the uterus or prostate.

4. An apparatus according to any preceeding claim wherein the housing is adapted to abut the foot of the subject adjacent the reproductive reflexology point of the pelvic musculature.

5. An apparatus according to any preceeding claim wherein the housing is adapted to abut the reproductive reflexology point of a foot of the subject corresponding to the ovaries or testes thereof.

6. An apparatus according to any preceeding claim wherein the housing is adapted to abut the calcaneal branches of the posterior tibial nerve of a foot of a subject.

7. An apparatus according to any preceeding claim wherein the housing is adapted to abut the sural nerve of a foot of a subject.

8. An apparatus according to any preceeding claim wherein the housing is adapted to abut more than one of the reproductive reflexology points, the calcaneal branches of the posterior tibial nerve and the sural nerve of the heel of a subject.

9. An apparatus according any preceeding claim wherein the housing of the device is adapted to abut both sides of the ankle.

10. An apparatus according to any preceeding claim adapted to vibrate against the heel of the foot of a subject to simultaneously co-stimulate the sural and calcaneal branches of the posterior tibial nerves.

11. An apparatus according to any preceeding claim wherein the housing is of U-shape construction wherein a portion of the housing is adapted to extend across the heel of a foot of the subject.

12. An apparatus according to any preceeding wherein at least one portion of the housing is cup or domed shaped to accommodate the round of the heel.

13. An apparatus according to claim 12 wherein the domed shaped portion of the housing comprises an annular rimmed portion for sealing to the round of the heel of a subject.

14. An apparatus according to claim 13 wherein the annular rimmed portion may comprise a vibration means.

15. An apparatus according to any preceeding claim wherein the housing comprises a resilient material to accommodate different widths of feet of different subjects.

16. An apparatus according to any preceeding claim wherein an outer surface of the housing is covered by a cover layer comprising relatively soft material.

17. An apparatus according to claim 16 wherein at least the portions of the housing which are to abut the foot of the subject are provided with the cover layer.

18. An apparatus according to claims 16 or 17 wherein the cover layer comprises at least one of a fabric material and a plastics material.

19. An apparatus according to claim 17 or claim 18 wherein the covering is decorated with various embellishments or attractive aesthetically pleasing designs.

20. An apparatus according to any preceeding claim wherein the housing comprises at least one arm member for gripping the foot of the subject at a position just below the ankle bone at the site of the reproductive reflex.

21. An apparatus according to claim 20 wherein the at least one arm member may be made of a flexible material.

22. An apparatus according to claim 21 wherein the at least one arm members may comprise vibrating means for vibrating the arm members at the site of the foot containing the reproductive reflexology point for transferring vibrations to the sites thereof to stimulate the reproductive reflexology point and/or the sural nerve positions.

23. An apparatus according to claim 22 wherein a battery is provided for powering the vibrating means.

24. An apparatus according to claim 23 wherein the battery is housed within the housing or is located externally to device.

25. An apparatus according to any preceding claim wherein a securing means is provided for securing the housing on a foot of the subject.

26. A kit comprising a pair of the apparatus is provided, one apparatus being provided for each foot of the subject.

27. A method for stimulating the libido of a subject and increasing feelings of wellbeing comprising vibrating more than one of a site selected from the group consisting of: the reproductive reflexology points, the calcaneal branches of the posterior tibial nerve and the sural nerve of the heel of a foot of a subject.

28. A method according to claim 27 wherein the site is vibrated by applying a vibrating means to the site.

29. A method according to claim 27 or 28 wherein the reproductive reflexology point is stimulated using the apparatus according to the invention.

30. An apparatus substantially as hereinbefore described with reference to the accompanying figures.
